(19) United States
(12) Patent Application Publication
(10) Pub. No.: US 2017/0096497 A1
(43) Pub. Date: Apr. 6, 2017

(54) USE OF ANTIBODY CONJUGATES

(71) Applicant: The Regents of the University of California, Oakland, CA (US)

(72) Inventor: Richard H. Weisbart, Los Angeles, CA (US)

(21) Appl. No.: 15/266,919

(22) Filed: Sep. 15, 2016

Related U.S. Application Data
(63) Continuation of application No. 12/523,438, filed on Jan. 26, 2010, now abandoned.
(60) Provisional application No. 60/897,012, filed on Jan. 22, 2007.

(51) Int. Cl. C07K 16/44; A61K 38/17

(52) U.S. Cl. CPC C07K 16/44; A61K 47/48569; A61K 38/1758; C07K 2317/14; C07K 2317/622; C07K 2317/73; C07K 2317/77; C07K 2317/92; C07K 2319/30

(57) ABSTRACT

Provided herein are methods for inducing growth arrest or apoptosis in cancer cells in a subject. Further provided are methods of inhibiting or treating metastasis of a cancer cell in a subject. The methods involve administering to the subject an antibody conjugate containing an antibody, variant thereof, or functional fragment thereof having binding specificity of the antibody as produced by the hybridoma having ATCC accession number PTA 2439 and a biologically active molecule. The antibody (e.g., mAb 3E10) variant or functional fragment thereof provides for the in vivo transduction of the conjugate to the nucleus of mammalian cells, where the conjugated biologically active molecule may exert its effect. In particular embodiments, the antibody conjugate comprises a single chain Fv fragment of an antibody having the binding specificity of mAb 3E10 produced by ATCC PTA 2439, conjugated to p53.

### RELATED APPLICATION

This application is a continuation of U.S. patent application Ser. No. 12/523,438, filed Jan. 26, 2010, which is a national phase of International PCT Application No. PCT/US08/51734, filed Jan. 22, 2008, which relies for priority under 35 U.S.C. §119(e) on U.S. Ser. No. 60/897,012, filed Jan. 22, 2007, which is incorporated herein by reference.

### INCORPORATION OF SEQUENCE LISTING

The material in the accompanying sequence listing is hereby incorporated by reference into this application. The accompanying sequence listing text file, name UCLA1690_2_ST25.txt, was created on 15 Sep. 2016, and is 11.7 kb. The file can be assessed using Microsoft Word on a computer that uses Windows OS.

### FIELD OF THE INVENTION

The present invention relates generally to the treatment of cancer and more specifically to the use of antibody conjugates to deliver biologically active compounds to cancer cells.

### BACKGROUND OF THE INVENTION

Missing or defective cellular proteins, such as p53 in many cancer cells, may be replaced via gene therapy or protein therapy. Gene therapy relies on the capacity of a cell to synthesize protein by using information encoded on exogenously provided DNA. Numerous viral and nonviral DNA delivery vectors have been tested, and p53 gene therapy has met with varying degrees of success both in vitro and in vivo. The primary factors limiting gene therapy at present include concerns over potential vector toxicity and immunogenicity, inefficient delivery of genes to cells, and relative instability of the transgene resulting in limited expression. As a potential alternative to gene therapy, protein therapy involves direct delivery of protein to the cells.

Protein therapy is defined as the direct delivery of therapeutic proteins into cells and tissues in order to treat or modify a disease process. Thus, protein therapy avoids certain hurdles of gene therapy, such as the expression of the exogenous gene and synthesis of a new protein and the need for a viral vector. Protein therapy does, however, face certain technical obstacles, such as the phospholipid bilayer of the cell membrane which excludes most proteins and peptides. However, novel protein transduction domains (PTDs) have been shown to be capable of crossing the plasma membrane. Such PTDs are peptides, proteins, or fragments of proteins that carry cargo proteins into cells in an apparently receptor-independent manner. PTDs described in the art include the HIV Tat peptide, polyarginine peptides, and the anti-DNA autoantibody monoclonal antibody (mAb 3E10).

The protein p53, often referred to as the guardian of the genome, plays a critical role in tumor suppression. Defects in p53 are linked to >50% of human cancers, and numerous studies have shown that restoring p53 function to p53-deficient cancer cells induces growth arrest and apoptosis. Various delivery vehicles have been used to deliver p53 and p53 peptides into cancer cells for restoration of p53 function. These include VP 22, a herpes simplex virus 1 protein, and the third alpha helix of Antennapedia homeodomain. The potential disadvantage of these vectors is that they are foreign proteins that may be immunogenic in humans. Developing a method to safely and efficiently restore p53 activity to tumor cells in vivo has become a key goal in cancer research.

Functional p53 or p53 peptides have been delivered to cancer cells in vitro using PTDs, such as the HIV Tat peptide, polyarginine peptides, and single-chain Fv of mAb 3E10. However, there have been no reports of successful full-length p53 protein therapy in vivo.

### SUMMARY OF THE INVENTION

The present invention is based on the discovery that the antibody conjugate Fv-p53 selectively kills cancer cells. Moreover, Fv-p53 effectively induces cell death in cancer cells with a variety of defects in p53, including absence of p53, mutations in p53, nuclear exclusion of p53, and overexpression of MDM2. As provided herein, invention methods were evaluated and found to be effective in preventing metastasis of colon carcinoma cells to the liver.

According to one embodiment of the invention, there are provided methods for inducing growth arrest or apoptosis in cancer cells in a subject. The method includes administering to the subject an antibody conjugate containing an antibody, variant thereof, or functional fragment thereof having binding specificity of an antibody as produced by the hybridoma having ATCC accession number PTA 2439 and a biologically active molecule, wherein the biologically active molecule is capable of inducing growth arrest or apoptosis. Without being bound to a particular theory, the antibody conjugate is transported into the cancer cell, thereby inducing growth arrest or apoptosis in the cancer cell. In one embodiment, the antibody is antibody mAb 3E10 as produced by the hybridoma having ATCC accession number PTA 2439 or a functional fragment or variant thereof or an antibody having the specificity of mAb 3E10. In one embodiment, the functional fragment is an scFv or Fab fragment. In further embodiments, the biologically active molecule is a p53 protein, peptide, or fragment thereof, or a full-length p53 protein. In other embodiments, the p53 protein or peptide is derived from a human p53 sequence. In certain embodiments, the cancer cell is p53-deficient or p53-defective.

According to another embodiment of the invention, there are provided methods for inhibiting or treating metastasis in a subject. The method includes administering to the subject an antibody conjugate containing an antibody, variant thereof, or functional fragment thereof having binding specificity of an antibody as produced by the hybridoma having ATCC accession number PTA 2439 and a biologically active molecule, wherein the biologically active molecule is capable of inhibiting or treating metastasis. In one embodiment, the antibody is antibody mAb 3E10 as produced by the hybridoma having ATCC accession number PTA 2439 or an functional fragment or variant thereof In another embodiment, the functional fragment is an scFv fragment. In further embodiments, the biologically active molecule is a p53 protein, peptide, or fragment thereof, preferably a full-length p53 protein. In other embodiments, the p53 protein or peptide is derived from a human p53 sequence. In certain embodiments, the cancer cell is p53-deficient or p53-defective.

According to another embodiment of the invention, there are provided methods for restoring p53 function in p53-deficient or p53-defective cancer cells in a subject. The method includes administering to the subject an antibody conjugate containing an antibody, variant thereof, or functional fragment thereof having binding specificity of an antibody as produced by the hybridoma having ATCC accession number PTA 2439 and a biologically active molecule, wherein the biologically active molecule is capable of restoring p53 function. In one embodiment, the antibody is antibody mAb 3E10 as produced by the hybridoma having ATCC accession number PTA 2439 or an functional fragment or variant thereof. In another embodiment, the functional fragment is an scFv fragment. In further embodiments, the biologically active molecule is a p53 protein, peptide, or fragment thereof, preferably a full-length p53 protein. In one embodiment, the p53 protein or peptide is derived from a human p53 sequence. In certain aspects, the restoration of p53 function results in growth arrest, cell cycle arrest, induction of apoptosis, or inhibition or treatment of metastasis.

### DESCRIPTION OF THE FIGURES

FIGS. 1A-C are graphs showing cytotoxicity of Fv-p53 in vitro in Skov-3 cells (FIG. 1A) and CT26.CL25 (FIG. 1B). FIG. 1C is a plot showing the dose response of the cytotoxic effect of Fv-p53 (nM) in CT26.CL25 cells.

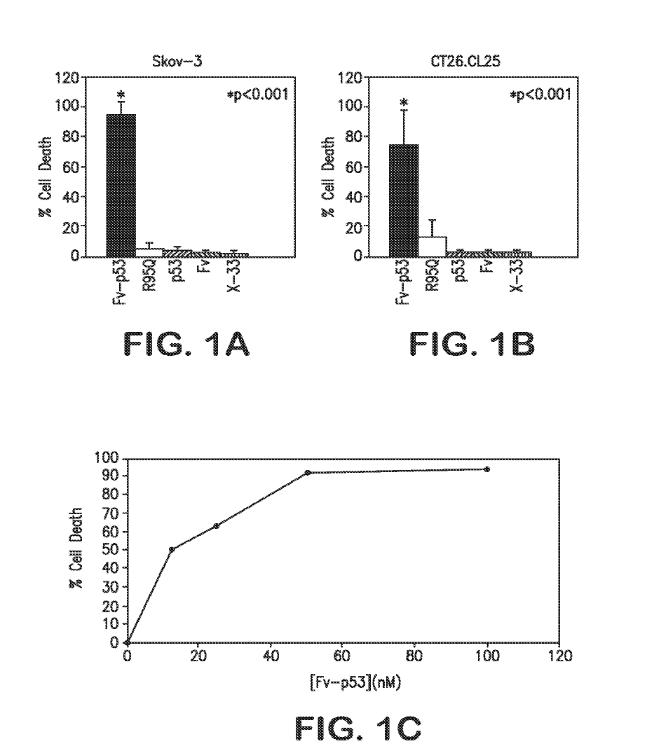

FIG. 2 shows the nucleotide sequence (SEQ ID NO:10; GenBank Accession NO. L16982) and amino acid sequence (SEQ ID NO:11) of mAb 3E10 V_{H}.

FIG. 3 shows the nucleotide and amino acid sequences of mAb 3E10 Vk light chains, 3E10VkIII (GenBank Accession No. L34051; SEQ ID NOs:12 and 13, for nucleotide and amino acid sequences, respectively) and 3E10VkSER (GenBank Accession No. L16983; SEQ ID NOs: 14 and 15, for nucleotide and amino acid sequences, respectively).

FIGS. 4A-B show the nucleotide (FIG. 4A) and amino acid (FIG. 4B) sequences for p53 (AAA61212 (SEQ ID NO:16) (encoded by open reading frame of the nucleotide sequence set forth in GenBank Accession No. M14695 (SEQ ID NO:17)).

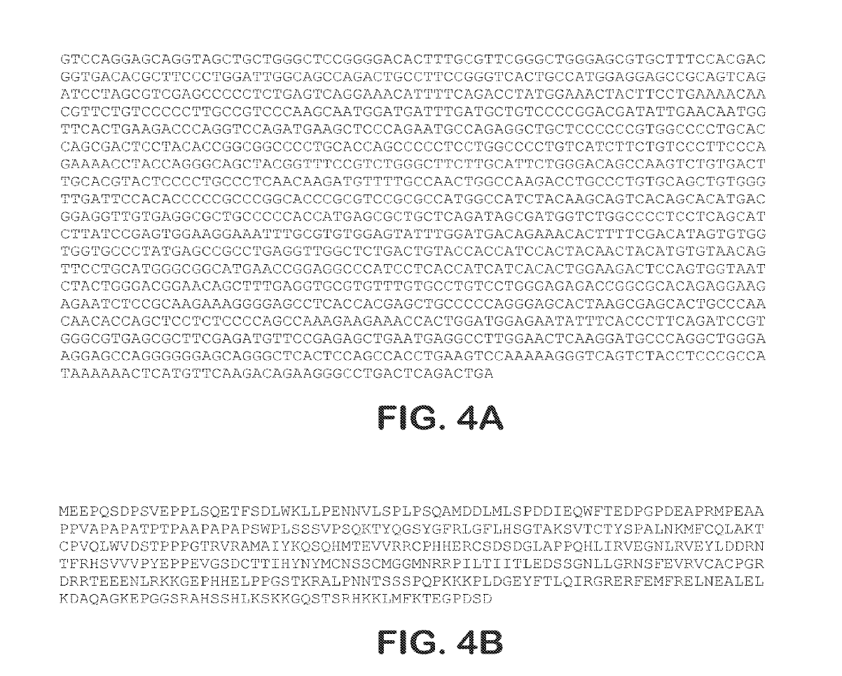

### DETAILED DESCRIPTION OF THE INVENTION

Before the present methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs.

In accordance with the present invention, there are provided methods for inducing growth arrest or apoptosis in cancer cells in a subject. The method includes administering to the subject an antibody conjugate containing an antibody, variant thereof, or functional fragment thereof having binding specificity of an antibody as produced by the hybridoma having ATCC accession number PTA 2439 and a biologically active molecule, wherein the biologically active molecule is capable of inducing growth arrest or apoptosis. It is believed that the antibody conjugate is transported into the cancer cell where the biologically active molecule can induce growth arrest or apoptosis in the cancer cell.

Also provided are methods for inhibiting or treating metastasis of cancer cells in a subject. The method includes administering to the subject an antibody conjugate containing an antibody, variant thereof, or functional fragment thereof having binding specificity of an antibody as produced by the hybridoma having ATCC accession number PTA 2439 and a biologically active molecule, wherein the biologically active molecule is capable of inducing growth arrest or apoptosis.

Cancer cells targeted by the invention methods may be from a cancer selected from the group consisting of colorectal cancer, esophageal cancer, stomach cancer, leukemia, lymphoma, lung cancer, prostate cancer, uterine cancer, skin cancer, endocrine cancer, urinary cancer, pancreatic cancer, other gastrointestinal cancer, ovarian cancer, cervical cancer, head and neck cancer, bone cancer, kidney cancer, liver cancer, bladder cancer, breast cancer, and adenomas. In certain embodiments, the cancer is colon cancer or ovarian cancer. In certain embodiments, cancer cells targeted by the invention methods may be p53-deficient or p53-defective or the status of p53 may be unknown. In other embodiments, the cancer cells targeted may contain a wild type p53.

Further provided are methods for restoring p53 function in p53-deficient or p53-defective cancer cells in a subject. The method includes administering to the subject an antibody conjugate containing an antibody, variant thereof, or functional fragment thereof having binding specificity of an antibody as produced by the hybridoma having ATCC accession number PTA 2439 and a biologically active molecule, wherein the biologically active molecule is capable of restoring p53 function. The antibody conjugate is transported into the p53-deficient or p53-defective cancer cell where the biologically active molecule restores p53 function. In some embodiments, the restoration of p53 function results in growth arrest, cell cycle arrest, induction of apoptosis, or inhibition or treatment of metastasis.

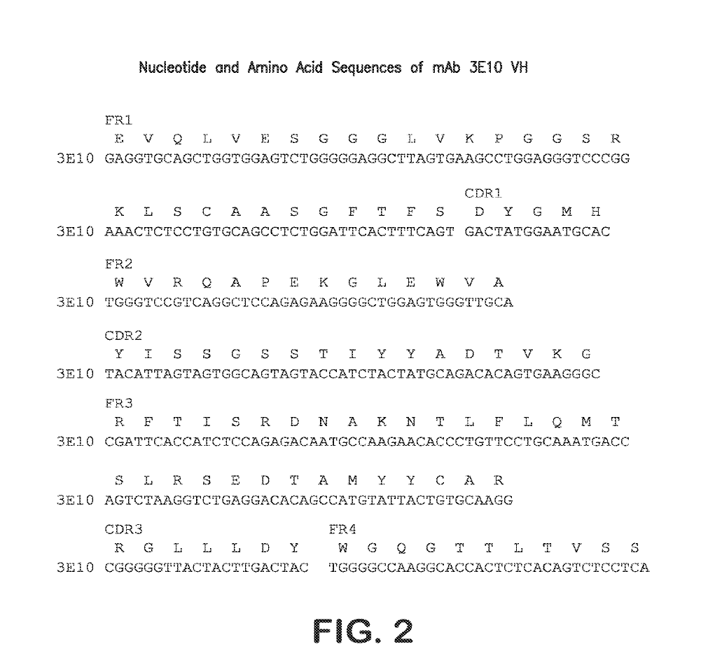

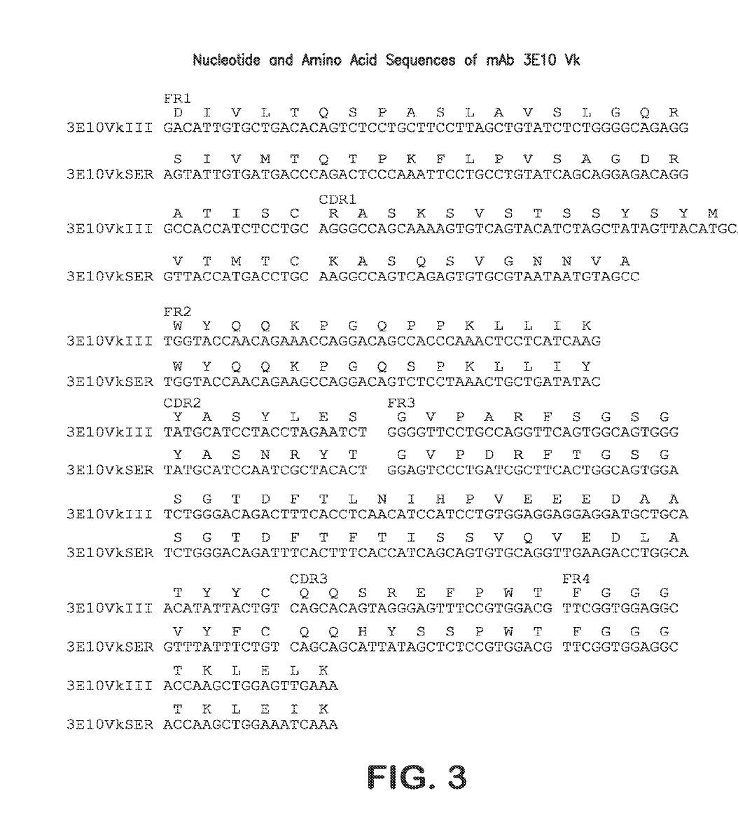

A class of DNA-binding autoantibodies can be utilized to transport a wide variety of biologically important molecules into target cells, such as kidney cells, brain cells, ovarian cells, hone cells, and the like. Examples of such DNA-binding autoantibodies include an antibody having the binding specificity of the antibody as produced by the hybridoma having ATCC accession number PTA 2439, antibody mAb 3E10, and variants and/or functional fragments thereof. The nucleotide and amino acid sequences for the variable region of the heavy chain of mAb 3E10 are provided in FIG. 2. The nucleotide and amino acid sequences for the variable region of the light chains of mAb 3E10 are provided in FIG. 3. In particular, the light chain designated VkIII contains the DNA binding capability for mAb 3E10. Thus, VkIII is the preferred light chain for 3E10 to be used in the methods of the present invention.

Although antibodies that penetrate living cells are frequently toxic or injurious and may explain some of the pathologic manifestations of the autoimmune diseases in which they are found, antibody mAb 3E10, in contrast, shows no harm to cells that it penetrates in tissue culture. Moreover, studies in vitro have shown that mAb 3E10 and scFv fragments of mAb 3E10 can transport relatively large proteins, such as catalase, into the nucleus of cells in tissue culture. Moreover, mAb 3E10 or fragments thereof (e.g., Fv) should not generate significant inflammation in vivo which could hinder therapeutic efficacy of a biologically active molecule conjugated thereto. Monoclonal antibody 3E10 is produced by the hybridoma 3E10 placed permanently on deposit with the American Type Culture Collection, 10801 University Blvd., Manassas, Va. 20110-2209, USA, on Aug. 31, 2000, according to the terms of the Budapest Treaty under ATCC accession number PTA-2439 and are thus maintained and made available according to the terms of the Budapest Treaty. Availability of such strains is not to be construed as a license to practice the invention in contravention of the rights granted under the authority of any government in accordance with its patent laws.

As used herein, “specific binding” refers to antibody binding to a predetermined antigen. Typically, the antibody binds with an affinity corresponding to a K_{D} of about 10⁻⁸ M or less, and binds to the predetermined antigen with an affinity (as expressed by K_{D}) that is at least 10 fold less, and preferably at least 100 fold less than its affinity for binding to a non-specific antigen (e.g., BSA, casein) other than the predetermined antigen or a closely-related antigen. Alternatively, the antibody can bind with an affinity corresponding to a K_{A} of about 10⁶ M⁻¹, or about 10⁷ M⁻¹, or about 10⁸ M⁻¹, or 10⁹ M⁻¹ or higher, and binds to the predetermined antigen with an affinity (as expressed by K_{A}) that is at least 10 fold higher, and preferably at least 100 fold higher than its affinity for binding to a non-specific antigen (e.g., BSA, casein) other than the predetermined antigen or a closely-related antigen. In some embodiments the antibody variant or functional fragment will have the same K_{A} or K_{D} as an antibody produced by the hybridoma having ATCC accession number PTA 2439. In certain embodiments, the antibody variant or functional fragment will have the same K_{A} or K_{D} as mAb 3E10.

The term “k_{d}” (sec⁻¹), as used herein, is intended to refer to the dissociation rate constant of a particular antibody-antigen interaction. This value is also referred to as the k_{off} value.

The term “kₐ” (M⁻¹ sec⁻¹), as used herein, is intended to refer to the association rate constant of a particular antibody-antigen interaction. The term “K_{A}” (M), as used herein, is intended to refer to the association equilibrium constant of a particular antibody-antigen interaction.

The term “K_{D}” (M⁻¹), as used herein, is intended to refer to the dissociation equilibrium constant of a particular antibody-antigen interaction.

Antibodies for use in the antibody conjugates of the present methods include an antibody having the binding specificity of the antibody as produced by the hybridoma having ATCC accession number PTA 2439, antibody mAb 3E10, and variants and/or functional fragments thereof. Such antibodies, variants or functional fragments thereof can be conjugated to the biologically active molecule of interest to form an antibody conjugate that is capable of being transported into the cell. Upon entry into the cell, it is believed that the antibody conjugate localizes in and around the cell nucleus. Antibody conjugates in accordance with the present invention may be used in the same manner as other conjugated delivery systems where an antibody or other targeting vehicle is conjugated to the biological molecule of interest to provide delivery to desired cells in the in vivo or in vitro environment.

Naturally occurring antibodies are generally tetramers containing two light chains and two heavy chains. Experimentally, antibodies can be cleaved with the proteolytic enzyme papain, which causes each of the heavy chains to break, producing three separate subunits. The two units that consist of a light chain and a fragment of the heavy chain approximately equal in mass to the light chain are called the Fab fragments (i.e., the “antigen binding” fragments). The third unit, consisting of two equal segments of the heavy chain, is called the Fc fragment. The Fc fragment is typically not involved in antigen-antibody binding, but is important in later processes involved in ridding the body of the antigen.

As used herein, the phrase “functional fragments of an antibody having the binding specificity of the antibody as produced by the hybridoma having ATCC accession number PTA 2439” refers to a fragment that retains the same cell penetration characteristics and binding specificity as mAb 3E10. Thus, in certain embodiments, a functional fragment of an antibody having the binding specificity of the antibody as produced by the hybridoma having ATCC accession number PTA 2439 or antibody mAb 3E10 is used in the antibody conjugate. In some embodiments, the functional fragment used in the antibody conjugate is selected from the group consisting of Fab, F(ab′)₂, Fv, and single chain Fv (scFv) fragments. In certain embodiments the functional fragment is an Fv fragments or an scFv fragment. In one example, the functional fragment includes at least the antigen-binding portion of mAb 3E10. In another example, the functional fragments is an scFv fragment comprising the variable region of the heavy chain (VH) and variable region of the kappa light chain (V_{K}) of mAb 3E10. For increased expression in the polynucleotide from which the scFv is expressed, the nucleic acids encoding the chains of mAb E310 are placed in reverse order with the V_{K} cDNA being placed 5′ of VH. In addition, one or more tags known in the art, preferably peptide (e.g., myc or His₆), may be incorporated into an antibody conjugate to facilitate in vitro purification or histological localization of the antibody conjugate. In some embodiments, the a myc tag and a His₆ tag are added to the C-terminus of VH.

As readily recognized by those of skill in the art, altered antibodies (e.g., chimeric, humanized, CDR-grafted, bifunctional, antibody polypeptide dimers (i.e., an association of two polypeptide chain components of an antibody, e.g., one arm of an antibody comprising a heavy chain and a light chain, or an Fab fragment comprising V_{L}, V_{H}, C_{L} and C_{H}1 antibody domains, or an Fv fragment comprising a V_{L} domain and a V_{H} domain), single chain antibodies (e.g., an scFv (i.e., single chain Fv) fragment comprising a V_{L} domain linked to a V_{H} domain by a linker, and the like) can also be produced by methods well known in the art. Such antibodies can also be produced by hybridoma, chemical synthesis or recombinant methods described, for example, in (Sambrook et al., Molecular Cloning: A Laboratory Manual 2d Ed. (Cold Spring Harbor Laboratory, 1989); incorporated herein by reference and Harlow and Lane, Antibodies: A Laboratory Manual (Cold Spring Harbor Laboratory 1988), which is incorporated herein by reference). Both anti-peptide and anti-antibody conjugate antibodies can be used (see, for example, Bahouth et al., Trends Pharmacol. Sci. 12:338 (1991); Ausubel et al., Current Protocols in Molecular Biology (John Wiley and Sons, N Y 1989) which are incorporated herein by reference). See in particular, FIGS. 2 and 3 for specific nucleotide and amino acid sequences of the illustrative antibody of the invention designated mAb 3E10.

For example, antibodies may be humanized by replacing sequences of the Fv variable region which are not directly involved in antigen binding with equivalent sequences from human Fv variable regions. General reviews of humanized chimeric antibodies are provided by Morrison et al., (Science 229:1202-1207, 1985) and by Oi et al. (BioTechniques 4:214, 1986). Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from for example, an antibody producing hybridoma. The recombinant DNA encoding the humanized or chimeric antibody, or fragment thereof, can then be cloned into an appropriate expression vector. Humanized antibodies can alternatively be produced by CDR substitution U.S. Pat. No. 5,225,539; Jones (1986) Nature 321:552-525; Verhoeyan et al. 1988 Science 239:1534; and Beidler (1988) J. Immunol. 141:4053-4060. Thus, in certain embodiments, the antibody used in the antibody conjugate is a humanized or CDR-grafted form of an antibody produced by the hybridoma having ATCC accession number PTA 2439. In other embodiments the antibody is a humanized or CDR-grafted form of antibody mAb 3E10. For example, the CDR regions of the illustrative antibody of the invention, as shown in FIGS. 2 and 3, can include amino acid substitutions such as 1, 2, 3, 4, 5, 6, 7, 8, 9, or 10 amino acid differences from those shown in the figures. In some instances, there are anywhere from 1-5 amino acid differences.

As used herein, reference to variants of an antibody having the binding specificity of an antibody as produced by the hybridoma having ATCC accession number PTA 2439″ includes variants retaining the same cell penetration characteristics and binding specificity as mAb 3E10, as well as variants modified by mutation to improve the utility thereof (e.g., improved ability to target specific cell types, improved ability to penetrate the cell membrane, improved ability to localize to the cellular DNA, and the like). Such variants include those wherein one or more conservative substitutions are introduced into the heavy chain, the light chain and/or the constant region(s) of the antibody. In some embodiments the variant has a light chain having an amino acid sequence at least 80% or at least 90% or at least 95% identical to the amino acid sequence set forth in SEQ ID NO:13. In other embodiments, the variant has a heavy chain having an amino acid sequence at least 80% or at least 90% or at least 95% identical to the amino acid sequence set forth in SEQ ID NO:11. Further, the invention includes antibodies that are encoded by nucleic acid sequences that hybridize under stringent conditions to the 3E10 variable region coding sequence (e.g., SEQ ID NO:10 and/or SEQ ID NO:12) or encode amino acid sequences at least 80% or at least 90% or at least 95% identical to the amino acid sequence set forth in SEQ ID NO:11 or SEQ ID NO:13.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50 degrees. C; (2) employ during hybridization a denaturing agent, such as formamide, for example, 50% (v/v) formamidc with 0.1% bovine serum albumin/0.1% Fico11/0.1% polyvinylpyrrolidone/50 mM sodium phosphate buffer at pH 6.5 with 750 mM sodium chloride, 75 mM sodium citrate at 42 degrees C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 .mu.g/ml), 0.1% SDS, and 10% dextran sulfate at 42 degrees C., with washes at 42 degrees C. in 0.2×SSC (sodium chloride/sodium citrate) and 50% formamide at 55 degrees C., followed by a high-stringency wash consisting of 0.1×SSC containing EDTA at 55 degrees C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37 degrees C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50 degrees C. The skilled artisan will recognize how to adjust the temperature, ionic strength, etc. as necessary to accommodate factors such as probe length and the like.

Such variants include those wherein one or more substitutions are introduced into the heavy chain nucleotide sequence, the light chain nucleotide sequence and/or the constant region(s) of the antibody. In some embodiments the variant has a light chain having a nucleotide sequence at least 80% or at least 90% or at least 95% identical to the nucleotide sequence set forth in SEQ ID NO:12. In other embodiments, the variant has a heavy chain having a nucleotide sequence at least 80% or at least 90% or at least 95% identical to the nucleotide sequence set forth in SEQ ID NO:10.

One exemplary variant contemplated for use in the practice of the present invention is an mAb 3E10 VH variant involving a single change of the aspartic acid residue at position 31 to asparagine (i.e., mAb 3E10-31). The preparation of this variant and further variants and a demonstration of its cell penetration ability is described in U.S. Pat. No. 7,189,396. This particular mAb 3E10 variant is especially well suited for delivery of biological molecules to kidney and brain cells. Other 3E10 variants and/or functional fragments thereof may be used to provide targeting of biologically active molecules. A wide variety of variants and/or functional fragments thereof are possible provided that they exhibit substantially the same cell penetration characteristics as mAb 3E10 or mAb 3E10-31 after conjugation to a selected biologically active molecule.

Antibodies according to the invention (e.g., mAb 3E10 and variants and/or functional fragments thereof) can be utilized to transport a wide variety of biologically active materials, e.g., nuclear transcription factors, enzymes, enzyme inhibitors, genes, and the like, to the cell nucleus for a variety of therapeutic effects. Pharmacologically active molecules including inorganic and organic molecules, pharmaceutical agents, drugs, peptides, proteins, genetic material, and the like, may be conjugated to antibodies according to the invention (e.g., mAb 3E10 and variants and/or functional fragments thereof) for delivery thereof

In some embodiments, Ab 3E10 heavy or light chains can be produced as antibody conjugates with a variety of biologically active molecules, e.g., nuclear transcription factors, enzymes, enzyme inhibitors, genetic material, inorganic or organic compounds, pharmaceutical agents, drugs, polypeptides and the like, thereby enabling the transport of these proteins into the cell nucleus of target cells. In addition, mAb 3E10 can be produced in the form of a fusion protein with other proteins that bind DNA (such as, for example, poly-L-lysine). The poly-L-lysine fusion protein with mAb 3E10 would bind DNA (e.g., plasmids containing genes of interest) and transport the DNA into the nucleus of target cells.

Antibody conjugates can be designed to place a polypeptide of interest at the amino or carboxy terminus of either the antibody heavy or light chain. Because the antigen binding fragments (Fab's) of mAb 3E10 have been shown to penetrate cells and localize in the nucleus, the entire heavy chain is not required. Therefore, potential configurations include the use of truncated portions of the heavy and light chain with or without spacer sequences as needed to maintain the functional integrity of the attached protein.

In addition to conjugating the antibody to the biologically active molecule, the latter can be attached to or associated with mAb 3E10 by any method known in the art. For example an scFv fragment of mAb 3E10, as described herein, can be expressed in a host cell as a fusion protein additionally containing a biologically active polypeptide for screening. Alternatively, the monoclonal antibody, or active fragment thereof, can be chemically linked to a polypeptide by a peptide bond or by a chemical or peptide linker molecule of the type well known in the art. In certain embodiments, a biologically active polypeptide is linked to the mAb 3E10 or fragment thereof via a peptide linker. The linker may be one or more tags (e.g., myc or His₆) or may be one or more repeats of the known linker sequence GGGGS (SEQ ID NO:1). Additional peptide linkers are known in the art. The skilled artisan will recognize that the linker sequence may be varied depending on the polypeptide to be linked to the antibody.

Methods for attaching a drug or other small molecule pharmaceutical to an antibody fragment are well-known and include bifunctional chemical linkers such as N-succinimidyl (4-iodoacetyl)-aminobenzoate; sulfosuccinimidyl(4-iodoacetyl)-aminobenzoate; 4-succinimidyl-oxycarbonyl-α-(2-pyridyldithio) toluene; sulfosuccinimidyl-6-[α-methyl-α-(pyridyldithiol)-toluamido] hexanoate; N-succinimidyl-3-(-2-pyridyldithio)-proprionate; succinimidyl-6[3(-2-pyridyldithio)-proprionamido] hexanoate; sulfosuccinimidyl-6-[3(+2-pyridyldithio)-propionamido] hexanoate; 3-(2-pyridyldithio)-propionyl hydrazide, Ellman's reagent, dichlorotriazinic acid, S-(2-thiopyridyl)-L-cysteine, and the like. Further bifunctional linking molecules are disclosed in U.S. Pat. Nos. 5,349,066; 5,618,528; 4,569,789; 4,952,394; and 5,137,877, each of which is incorporated herein by reference in its entirety.

As used herein, the phrase “biologically active molecule” refers to a molecule that has a biological effect in a cell. Exemplary biologically active molecules include a nuclear transcription factor, an enzyme inhibitor, genetic material, an inorganic or organic molecule, a pharmaceutical agent, a drug, or a polypeptide. In certain embodiments, the biologically active molecule is a polypeptide.

In particular embodiments, the biologically active molecule is a p53 protein or peptide fragment thereof p53 is the protein product of the tumor suppressor gene TP53 and plays critical and complicated roles in cell cycle regulation and protection against the development of cancer. p53 responds to abnormalities in the normal cellular milieu by initiating cell cycle arrest and inducing apoptosis if the cell is unable to repair the damage and restore normal functioning. Events known to activate p53 include DNA damage, oxidative stress, and hypoxia. A cell that fails to repair mutated DNA after p53 has signaled a halt in cell cycle progression will eventually enter apoptosis through p53-mediated activation of transcription of pro-apoptotic genes or by a direct interaction of p53 with the mitochondria.

The capacity of p53 to induce apoptosis in cells that have suffered genomic damage is critical to the prevention of cancer. Without the constant surveillance of the cell by p53, mutated cells are not removed from the tissues and instead, accumulate through repeated cycles of cell division, ultimately resulting in tumor growth. Cells and organisms deficient in p53 are predisposed to the accumulation of mutations and development of cancer.

The phrase “p53 deficient” as used herein refers to a decreased level, or the absence of p53 protein in the cell. In addition, a p53-deficiency may be the result of p53 being prevented from carrying out its normal function by nuclear exclusion or over-expression of internal cellular elements such as MDM2, a negative regulator of p53. The phrase “p53 defective” as used herein refers to a cell having a mutated p53 or an improperly post-translationally modified p53, resulting in an impairment of p53 function.

Thus, in certain embodiments of the present methods, p53 is delivered to cancer cells via an antibody conjugate, where p53 is localized to in or around the nucleus and can exert its biological affect. In some embodiments p53 is a full-length molecule, preferably human p53. An exemplary human p53 sequence is provided in GenBank Accession No. AAA61212 (encoded by open reading frame of the nucleotide sequence set forth in GenBank Accession No. M14695). The skilled artisan would however recognize that p53 proteins from other species, preferably mammalian, may be used provided such proteins are substantially similar to the human sequence or have been modified so as to not elicit an unfavorable immune response. In particular embodiments, a full length human p53 is conjugated to scFv mAb 3E10. In one embodiment, the antibody conjugate is an scFv mAb 3E10 fusion protein. In certain embodiments, a p53 peptide may be used in an antibody conjugate. For example, certain p53 peptides, such as the C-terminal 30 amino acids of p53 have demonstrated a cytotoxic effect when delivered to SW480 cancer cells, which harbor a mutant p53 (U.S. Pat. No. 7,189,396).

In other embodiments, the polypeptide may be an antibody, preferably a monoclonal antibody. In one example, the antibody is an anti-p53 antibody. One example of an anti-p53 antibody is mAb PAb421 which binds the C-terminal portion of p53 (Weisbart et al., Int J Oncology 25:1113-8, 2004).

Antibody conjugates may be produced by recombinant methods well-known in the art. For example, an antibody conjugate comprising a biologically active polypeptide may be produced as a fusion protein using recombinant methods to construct a polynucleotide encoding the fusion protein. The polynucleotide may be constructed so that the fusion protein contains linker or tag sequences. The polynucleotide encoding an antibody conjugate can be ligated into an expression vector. The vector may further comprise expression regulatory sequences operably associated with the polynucleotide that can control and regulate the production in an appropriate host cell of a polypeptide(s) encoded by the polynucleotide.

Vectors suitable for use in preparation of polypeptides such as the antibody conjugate include those selected from baculovirus, phage, plasmid, phagemid, cosmid, fosmid, bacterial artificial chromosome, viral DNA, Pl-based artificial chromosome, yeast plasmid, and yeast artificial chromosome. For example, the viral DNA vector can be selected from vaccinia, adenovirus, foul pox virus, pseudorabies and a derivative of SV40. Suitable bacterial vectors for use in practice of the invention methods include pQE70, pQE60, pQE-9, pBLUESCRIPT SK, pBLUESCRIPT KS, pTRC99a, pKK223-3, pDR540, PAC and pRIT2T. Suitable eukaryotic vectors for use in practice of the invention methods include pWLNEO, pXTI, pSGS, pSVK3, pBPV, pMSG, and pSVLSV40. Suitable eukaryotic vectors for use in practice of the invention methods include pWLNEO, pXTI, pSG5, pSVK3, pBPV, pMSG, and pSVLSV40.

Those of skill in the art can select a suitable regulatory region to be included in such a vector, for example from lacI, lacZ, T3, T7, apt, lambda PR, PL, trp, CMV immediate early, HSV thymidine kinase, early and late SV40, retroviral LTR, and mouse metallothionein-I regulatory regions.

Host cells in which the vectors containing the polynucleotides can be expressed include a bacterial cell, a eukaryotic cell, a yeast cell, an insect cell, or a plant cell. For example, E. coli, Bacillus, Streptomyces, Pichia pastoris, Salmonella typhimurium, Drosophila S2, Spodoptera SJ9, CHO, COS (e.g. COS-7), or Bowes melanoma cells are all suitable host cells for use in practice of the invention methods.

In other embodiments, the biologically active molecule is a polynucleotide. A polynucleotide, such as one encoding a therapeutic protein, can be delivered to cancer cells by chemically bonding the polynucleotide to an antibody or fragment as disclosed herein, such as mAb 3E10 of a function fragment thereof, for example an scFv or Fab. Polynucleotides delivered into cancer cells in the subject using the antibody conjugate may become stably integrated into the nucleus of the cancer cells. If the polynucleotide contains a gene rather than a regulatory molecule, the gene can be expressed in the cancer cells of the subject.

Pharmaceutical compositions comprising an antibody conjugate may be used in the methods described herein. Thus, in one embodiment, a pharmaceutical composition including a antibody conjugate present in an amount effective to induce growth arrest or apoptosis in cancer cells in a subject is used in methods described herein. In another embodiment, a pharmaceutical composition including a antibody conjugate present in an amount effective to inhibit or treat metastasis of cancer cells in a subject is used in methods described herein. In a further embodiment, a pharmaceutical composition including a antibody conjugate present in an amount effective to restore p53 function to cancer cells in a subject is used in methods described herein. In addition to the antibody conjugate, the pharmaceutical composition may also contain other therapeutic agents, and may be formulated, for example, by employing conventional vehicles or diluents, as well as pharmaceutical additives of a type appropriate to the mode of desired administration (for example, excipients, preservatives, etc.) according to techniques known in the art of pharmaceutical formulation.

The term “effective amount” of a compound refers an amount that is non-toxic to a subject or a majority or normal cells, but is an amount of the compound that is sufficient to provide a desired effect (e.g., inhibition of metastasis of a melanoma, sensitization of cells to apoptosis, induction of cell growth or cell cycle arrest, induction of apoptosis, or restoration of p53 function). This amount may vary from subject to subject, depending on the species, age, and physical condition of the subject, the severity of the disease that is being treated, the particular antibody conjugate, or more specifically, the particular biologically active molecule used, its mode of administration, and the like. Therefore, it is difficult to generalize an exact “effective amount,” yet, a suitable effective amount may be determined by one of ordinary skill in the art.

The term “pharmaceutically acceptable” refers to the fact that the carrier, diluent or excipient must be compatible with the other ingredients of the formulation and not deleterious to the recipient thereof. For example, the carrier, diluent, or excipient or composition thereof may be administered to a subject along with an antibody conjugate of the invention without causing any undesirable biological effects or interacting in an undesirable manner with any of the other components of the pharmaceutical composition in which it is contained.

Pharmaceutical compositions comprising the antibody conjugate may be administered by any suitable means, for example, parenterally, such as by subcutaneous, intravenous, intramuscular, intrathecal, or intracisternal injection or infusion techniques (e.g., as sterile injectable aqueous or non-aqueous solutions or suspensions) in dosage formulations containing non-toxic, pharmaceutically acceptable vehicles or diluents. In certain embodiments the antibody conjugate is administered parenterally, or more preferably, intraveneously.

The mode of delivery chosen for administration of antibody conjugates according to the present invention to a subject, such as a human patient or mammalian animal, will depend in large part on the particular biologically active molecule present in the antibody conjugate and the target cells. In general, the same dosages and administration routes used to administer the biologically active molecule alone will also be used as the starting point for the antibody conjugate. However, it is preferred that smaller doses be used initially due to the expected increase in cellular penetration of the biological molecule. The actual final dosage for a given route of administration is easily determined by routine experimentation. In general the same procedures and protocols that have been previously used for other antibody-based targeting conjugates (e.g., parenterally, intravenous, intrathecal, and the like) are also suitable for the antibody conjugates of the present invention.

The pharmaceutical compositions of the antibody conjugate can be administered either alone or in combination with other therapeutic agents, may conveniently be presented in unit dose form and may be prepared by any of the methods well known in the art of pharmacy. All methods include bringing the antibody conjugate into association with the carrier, which constitutes one or more accessory ingredients. In general, the pharmaceutical compositions are prepared by uniformly and intimately bringing the active ingredient into association with a liquid carrier. In the pharmaceutical composition the antibody conjugate is included in an amount sufficient to produce the desired effect upon the process or condition of disease.

Depending on the condition being treated, these pharmaceutical compositions may be formulated and administered systemically or locally. Techniques for formulation and administration may be found in the latest edition of “Remington's Pharmaceutical Sciences” (Mack Publishing Co, Easton Pa.). Suitable routes may, for example, parenteral delivery, including intramuscular, subcutaneous, intramedullary, intrathecal, intraventricular, intravenous, or intraperitoneal. For injection, the pharmaceutical compositions of the invention may be formulated in aqueous solutions, preferably in physiologically compatible buffers such as Hanks' solution, Ringer's solution, or physiologically buffered saline.

The invention will now be described in greater detail by reference to the following non-limiting examples.

### Example 1

In the present study, in vitro experiments have been extended to include additional control proteins to verify that Fv-p53 is the factor responsible for cell killing in cancer cell lines. Furthermore, Fv-p53 protein therapy was tested in vivo and found it strikingly effective in preventing metastasis of colon carcinoma cells to the liver. Specifically, clinical efficacy of monoclonal antibody (mAb) 3E10 Fv antibody-mediated p53 protein therapy was evaluated by testing an Fv-p53 fusion protein produced in Pichia pastoris on CT26.CL25 colon cancer cells in vitro and in vivo in a mouse model of colon cancer metastasis to the liver. In vitro experiments showed killing of CT26.CL25 cells by Fv-p53, but not Fv or p53 alone, and immunohistochemical staining confirmed that Fv was required for transport of p53 into cells. Prevention of liver metastasis in vivo was tested by splenic injection of 100 nmol/L Fv-p53 at 10 min and 1 week after injection of CT26.CL25 cancer cells into the portal vein of BALB/c mice. The results indicate that Fv-p53 treatment had a profound effect on inhibition of liver metastasis and represent the first demonstration of effective full-length p53 protein therapy in vivo.

Plasmids

pPICZA-Fv-p53. cDNA encoding an Fv-p53 fusion protein was ligated into pPICZA as described previously (Weisbart et al., Int J Oncol 25:1867-73, 2004). Briefly, mAb 3E10 Fv cDNA containing 3′ myc and His₆ tags was amplified by PCR and ligated into the EcoRI and BamHI cites in pSG5 (Stratagene, La Jolla, Calif.) as a cassette for the construction of fusion proteins as previously described (Weisbart et al., Cancer Lett 195:211-19, 2003). p53 cDNA was amplified by PCR from pCD53 as described previously and included 5′-BamHI and 3′BglII restriction sites. The sense primer was 5′-GGATCCGAGGAGCCGCAGTCAGAT-3′ (SEQ ID NO:2) and the antisense primer was 5′-AGATCTTCAAATATCGTCCGGGGACAG-3′ (SEQ ID NO:3). The PCR fragment was ligated into PCR2.1 (Invitrogen Corp., Carlsbad, Calif.), excised with BamHI and BglII and ligated into pSG5 containing mAb 3E10 Fv cDNA to produce a fusion construct. The Fv-p53 cDNA construct was re-amplified by PCR to incorporate a 5′ yeast consensus sequence and change the 3′ restriction site to SacII for ligation into pPicZA for intracellular expression in Pichia pastoris. The sense primer was 5′-GAATTCGGGATGGACATTGTGCTGACAC-3′ (SEQ ID NO:4) and the antisense primer was 5′-CCGCGGTCAATGATGATGATGATGATGGTC-3′ (SEQ ID NO:5). The design of the construct was Fv-myc-His₆-p53.

pPICZA-Fv(R95Q)-p53. The pPICZA-Fv(R95Q)-p53 construct was generated by site-directed mutagenesis of the pPICZA-Fv-p53 construct using the QuikChange kit (Stratagene, La Jolla, Calif.) with mutagenesis primers 5′-CAGTAGTCAAGTAGTAACCCCTGCCTTGCACAG-3′ (SEQ ID NO:6) and 5′-CATGTATTACTGTGCAAGGCAGGGGTTACTACTT-3′ (SEQ ID NO:7).

pPICZA-p53. cDNA encoding wild-type p53 was PCR amplified from the pPICZA-Fv-p53 construct using sense primer 5′-GAATTCATGCATCATCATCATCATCATGAGGAGCGGCAGTCAG-3′ (SEQ ID NO:8) and antisense primer 5′-CTCGAGTCAGTCTGAGTCAGGCCC-3′ (SEQ ID NO:9). The PCR product was inserted into the pCR2.1 vector with use of the TA Cloning kit (Invitrogen, Carlsbad, Calif.). The p53 cDNA insert was liberated from pCR2.1-p53 by digestion with EcoRI and XhoI and ligated into EcoRI and XhoI sites in pPICZA.

pPICZαA-Fv. cDNA encoding the single-chain fragment of mAb 3E10 was ligated into pPICZαA as described previously (Int J Oncol 2004; 25:1713-8).

Recombinant Proteins

Fv-p53, Fv(R95Q)-p53, wild-type p53, Fv, and X-33 control proteins were produced in and purified from Pichia pastoris and analyzed by SDS-PAGE followed by Western blot analysis as described previously (Weisbart et al. Int J Oncol 25:1867-73, 2004). Typical yields of Fv-p53 and Fv(R95Q)-p53 were 30 μg from a 500 mL culture. Typical yields of wild-type p53 and Fv were 3 mg from a 500 mL culture. Concentrations of Fv-p53 were determined by an ELISA capture assay with anti-p53 antibodies and comparison with a standard curve.

Cell Lines

Skov-3 ovarian cancer and CT26.CL25 colon cancer cell lines were acquired from the American Type Culture Collection (Rockville, Md.).

Nuclear Penetration Assay

Fv-p53, Fv(R95Q)-p53, or wild-type p53 (100 nmol/L) was applied to Skov-3 cells. As a positive control, 100 μmol/L Fv was also applied to the cells. After 1 h of incubation, cells were washed, fixed, and stained with anti-p53 pAb421 or anti-myc antibodies as described previously (Weisbart et al. Int J Oncol 25:1867-73, 2004).

Microscopic Images

An Olympus IX70 inverted microscope with RC reflected light fluorescent attachment and MagnaFire SP Digital Imaging System (Olympus, Melville, N.Y.) was used to acquire microscopic images of cells as described previously (Weisbart et al., J Immunol 164:6020-6, 2000).

In Vitro Cytotoxicity Assay

Fv-p53, Fv(R95Q)-p53, wild-type p53, or Fv (100 nmol/L) was applied to Skov-3 and CT26.CL25 cells. Control cells were incubated with X-33 yeast proteins. Twenty-four hours after addition of proteins to the cells, percentage cell death was determined by propidium iodide staining as described previously (Weisbart et al. Int J Oncol 25:1867-73, 2004).

In Vivo Liver Metastasis Model

A “hemispleen” model, as first described by Schulick et al. (Ann Surg Oncol 10:810-20, 2003), was optimized. BALB/c mice at 10 weeks of age were purchased from The Jackson Laboratory (Bar Harbor, Me.). The fur on the left flank was removed using clippers. The animals were anesthetized using halothane, and the surgical area was prepped with povidone iodine. A 1.0 cm to 1.5 cm incision was made in the left flank, and the peritoneal cavity was entered. The stomach was gently grasped to bring the entire spleen into view. Two medium vascular clips (Week, Research Triangle Park, N.C.) were placed across the midbody of the spleen. The spleen was then divided between these clips, leaving two hemispleens, each with their own vascular pedicle. A 27-gauge needle was used to inject 1×10⁵ CT26.CL25 colon cancer cells into the inferior hemispleen. Before this injection, the syringes were preloaded with 250 μL HBSS. During the surgery, 50 μL of cell suspension were aspirated into the syringe, thus providing a saline flush after the cells were injected. Three minutes after the cell injection, a medium vascular clip was placed across the vascular pedicle and the inferior hemispleen was removed. Ten minutes later, the treatment or control solution was injected into the superior hemispleen in a similar manner. The hemispleen was left in place for a second injection 7 days later. The abdomen was then closed in a single layer using 5-0 Prolene suture. The animals were euthanized 2 weeks later, and the livers were examined. The whole liver was assigned a metastasis score of 0 (no gross metastasis), 1 (<1 cm² area of tumor), 2 (1-2 cm² area of tumor), 3 (>2 cm² area of tumor), or 4 (complete infiltration).

A “portal vein” model was also optimized (Cai et al., Int J Oncol 27:113-20, 2005). BALB/c mice at 10 weeks of age were used. The animals were prepped and anesthetized as described previously. An upper midline incision was made, and the peritoneal cavity was entered. The intestines were eviscerated and reflected to the right. A piece of warm saline-soaked gauze measuring 2×2 inches was placed over the intestines. A 31-gauge needle was used to inject 4×10⁵ CT26.CL25 colon cancer cells in 200 μL HBSS into the portal vein. A small piece of moist Gelfoam (Pharmacia Corp., Kalamazoo, Mich.) was then pressed over the injection site. Pressure was continued for 2 to 3 min, and the Gelfoam was left in place. The intestines were then returned to the abdomen, which was closed in one layer using 5-0 Prolene suture. The animal was then Q2 turned, and a second incision was made over the left flank. A small s.c. pocket was dissected, and then, the abdomen was entered. The whole spleen was used for injection of either Fv-p53 treatment or X-33 yeast protein control. After the injection, the whole spleen was placed into the s.c. pocket to facilitate subsequent injections. The spleen was held in position by closing the abdominal wall with 5-0 Prolene suture as described by Kasuya et al. (Cancer Res 65:3823-7, 2005). The skin was then closed in a separate layer using the same suture. A second spleen injection was done 7 days later via a minor surgery. The animal was anesthetized, and the left flank was prepped with povidone iodine. A small portion of the incision was opened, and the material was injected into the spleen under direct visualization. Seven days after the second injection, the animals were euthanized and a metastasis score (see above for criteria) was given to the left lobe of the liver that receives drainage from the splenic vein.

Statistics

P values were determined by using a two-tailed Student's t test.

The Fv Fragment is Required for Nuclear Delivery of p53.

Fv-p53, Fv(R95Q)-p53, p53 alone, Fv alone, and X-33 control proteins were generated and purified from P. pastoris as described previously. Fv(R95Q)-p53, abbreviated as R95Q, contains a mutation in Fv that renders the protein incapable of penetrating into the cells (Weisbart et al., Int Jr Oncol 25:1113-8, 2004). X-33 proteins were eluted from Ni-NTA agarose (Qiagen, Valencia, Calif.) incubated with lysates of X-33 cells free of plasmids. The X-33 control showed the same pattern of proteins found in preparations of Fv-p53 and served as a control for protein impurities that copurify with Fv-p53. Fv-p53 and control proteins were tested for penetration into Skov-3 cells. Control cells treated with X-33 yeast proteins showed an absence of staining. Cells treated with Fv or Fv-p53 exhibited distinct nuclear staining representing nuclear penetration. As expected, cells treated with R95Q or p53 alone did not show nuclear staining. Because p53 alone failed to penetrate into Skov-3 cells, these results indicate that the Fv fragment is necessary for nuclear delivery of p53.

Functional Fv-p53 is Required for Induction of Cell Death in Cancer Cells.

Induction of cell death in cancer cells by Fv-p53 was compared with control proteins by applying equimolar amounts of the proteins to Skov-3 and CT26.CL25 cells. Twenty-four hours after application of protein, the cells were analyzed under fluorescence microscopy using propidium iodide staining to quantify cell death. Skov-3 cells (93.8±8.9%) were killed by Fv-p53 compared with 5.2±4.3% by R95Q, 4.0±2.8% by p53, 3.0±1.4% by Fv, and 2.2±1.5% by X-33 proteins (FIG. 3A). Similarly, 74.6±24.1% of CT26.CL25 cells were killed by Fv-p53 compared with 13.8±10.9% by R95Q, 3.0±1.4% by p53, 2.5±0.7% by Fv, and 2.5±0.6% by X-33 proteins (FIG. 3B). Both wildtype p53 and the R95Q mutant fusion protein failed to penetrate into the cells and to kill Skov-3 or CT26.CL25 cells, indicating that transducible p53 is required for cell killing. Taken together, the penetration and killing assays indicate that Fv-p53 is the functional reagent responsible for killing the Skov-3 and CT26.CL25 cells.

To determine the concentration of Fv-p53 required for killing CT26.CL25 cells, the cell death assay was repeated using 12.5, 25, 50, and 100 nmol/L of Fv-p53. Both 50 and 100 nmol/L Fv-p53 were highly effective in killing the CT26.CL25 cells, whereas lower doses exhibited significantly less activity (FIG. 3C). This result showed that Fv-p53 has a dose-dependent effect on CT26.CL25 cells and suggested the concentration of Fv-p53 to be tested in vivo.

Fv-p53 Prevents Liver Metastasis In Vivo.

A liver metastasis model, generated by injecting CT26.CL25 colon carcinoma cells into BALB/c mice, was used to test the efficacy of Fv-p53 protein therapy in vivo. The first mouse experiment used the “hemispleen” method to optimize the timing of Fv-p53 delivery after injection of the cancer cells. CT26.CL25 colon carcinoma cells were given to 12 BALB/c mice, which were divided into four groups. Mice in each group received two hemispleen injections of 100 nmol/L Fv-p53 or control medium. The first injections of Fv-ip53 or control medium were made 10 min after administration of the CT26.CL25 cells, whereas the second injections occurred 1 week later. Mice in group 1 received control medium for both injections. Mice in group 2 received Fv-p53 for the first injection and control medium for the second injection. Group 3 mice received control medium for the first injection and Fv-p53 for the second injection. Finally, group 4 mice received Fv-p53 for both the first and second injections. Two weeks after the second injections, the mice were euthanized and the livers were examined to determine the extent of tumor burden. The mice in group 1 had an average metastasis score of 2.7±0.5. In contrast, group 4 had an average metastasis score of 1.0±0.0, indicating a decrease in tumor burden in the treated mice. Mice in groups 2 and 3 had scores of 0.7±0.9 and 2.0±0.8, respectively (Table 1). These results suggest that Fv-p53 seems to decrease the metastatic burden, particularly if given early. This shows that Fv-p53 seems to have an effect on the prevention of liver metastasis, and as expected, early treatment was more effective than delayed treatment.

TABLE 1
Optimization of Fv-p53 delivery

| | No. | Treatment | Treatment | Metastasis |
|---|---|---|---|---|
| Group | Mice | at 10 min | at 1 wk | Score* |
| 1 | 3 | Control | Control | 2.7 ± 0.5 |
| 2 | 3 | Fv-p53 | Control | 0.7 ± 0.9 |
| 3 | 3 | Control | Fv-p | 53 2.0 ± 0.8 |
| 4 | 3 | Fv-p53 | Fv-p53 | 1.0 ± 0.0 |

*Results are reported as mean ± SD.

In the first mouse experiment, significant local recurrence of tumor in the left upper quadrant of the abdomen near the spleen site was noted. Therefore, the “portal vein” method was used for the second experiment in an effort to decrease or eliminate the amount of local recurrence of tumor. In this experiment, liver metastases were established by injecting CT26.CL25 cells into the portal vein, and the mice were treated via splenic injection with either Fv-p53 or X-33 yeast protein control at 10 min and again 7 days later. The animals were euthanized 7 days after the second injection, and the livers were examined for tumor burden. Mice treated with Fv-p53 had a significantly lower metastasis score than mice treated with X-33 control (Table 2). A reduction in metastasis score from 3.3±1.3 to 0.8±0.4 represents a clinically significant decrease in tumor burden. Control mice had severe to complete infiltration of livers, whereas mice treated with Fv-p53 had minimal liver infiltration. Taken together, the two mouse experiments show that Fv-p53 inhibits liver metastasis and provides the first experimental evidence of effective full-length p53 protein therapy in vivo.

TABLE 2
Effects of Fv-p53 on the development of liver metastasis

| | No. | Treatment | Treatment | Metastasis |
|---|---|---|---|---|
| Group | Mice | at 10 min | at 1 wk | Score* |
| 1 | 3 | Control | Control | 2.7 ± 1.3 |
| 2 | 5 | Fv-p53 | Fv-p53 | 0.8 ± 0.4 |

Results are reported as mean ± SD.
*P = 0.004

Developing therapeutic agents that selectively kill cancer cells while sparing healthy cells and tissues will significantly increase the likelihood of achieving cure in cancer patients. Whereas solitary primary tumors may be amenable to surgical removal, metastatic disease is often incurable. Present efforts to treat metastatic disease primarily rely on radiation therapy and chemotherapeutic drugs, which may cause significant side effects. Monoclonal antibodies (mAbs) that bind specific tumor antigens, such as trastuzumab (Herceptin), typically have fewer side effects but usually have the greatest activity when used together with more toxic chemotherapy agents (Mehra et al., Expert Opin Biol Ther 6:951-62, 2006). p53 therapy presents a potentially elegant solution to the problem of metastatic disease in that small doses of p53 induce growth arrest and apoptosis in transformed cells but do not seem to adversely affect normal cells (Weisbart et al. Int J Oncol 25:1867-73, 2004). It also has the profound advantage of likely being applicable to greater than half of all tumor cells.

Protein therapy could be effective in treating nearly any protein deficiency disease but is particularly well suited to the treatment of cancer. Whereas patients with chronic diseases might require continuous replacement therapy, cancer patients would potentially require only a limited number of doses of p53 to eliminate cancer cells. Furthermore, p53 is functional at a very low intracellular concentration, and the frequency and duration of p53 infusions could be easily modified to minimize side effect profiles. The Fv fragment of mAb 3E10 is an ideal transport vehicle for p53 protein therapy. Fv specifically delivers most cargo proteins to the nucleus and should promote less inflammation than other protein transduction domains (PTDs) (El-Amine et al., Int Immunol 14:761-6, 2002; Zambidis et al., PNAS 93:5019-24, 1996). In the present study, no side effects of Fv-p53 therapy were observed in any of the experimental mice. Fv also has a short half-life inside the cells, and previous studies using single-chain Fv fragments to visualize tumors in vivo found that Fv fragments localize into the tumor cells more readily than normal cells and are rapidly cleared from the body (Yokota et al., Cancer Res 52:3402-8, 1992; Yokota et al., Cancer Res 53:3776-83, 1993; Erratum in: Cancer Res 53:5832, 1993). The propensity of Fv fragments to localize to tumors would facilitate delivery of p53 to target tissues, and if side effects become a concern, the short half-life of Fv and rapid plasma clearance of Fv fragments would aid in limiting therapy duration. The timing of Fv-p53 treatment in relationship to the development of metastatic disease seems to be important. In our mouse model, Fv-p53 given 10 min after injection of cancer cells seemed to suppress metastasis to the greatest extent (Table 1). Although the mice receiving Fv-p53 at 7 days seemed to have a larger metastatic burden compared with those receiving an initial dose of Fv-p53 at 10 min, there was still a suggestion that they had a lower metastasis score compared with those that received the control injection at both times. The ability of Fv-p53 to suppress metastasis whether given 10 min or 1 week after administration of cancer cells suggests that Fv-p53 may be able to kill both recently metastasized cells and established tumor cells.

The second in vivo experiment showed that Fv-p53 treatment had a profound effect on liver metastasis. Control mice treated with X-33 yeast proteins had severe to complete tumor infiltration of the liver, whereas mice treated with Fv-p53 had minimal to no liver metastasis. This result was not only statistically significant but also readily apparent on gross observation of the livers from control and Fv-p53-treated mice (Table 2). This shows that Fv-p53 has activity even in the setting of a very large metastatic burden. The primary significance of this work is that, for the first time, full-length p53 protein therapy is effective in vivo, suggesting the use of Fv-p53 protein therapy as an effective cancer therapy.

Although the invention has been described with reference to the above example, it will be understood that modifications and variations are encompassed within the spirit and scope of the invention. Accordingly, the invention is limited only by the following claims.

1-53. (canceled)

54. A method for inhibiting or treating metastasis of a cancer cell in a subject, comprising administering to the subject an antibody conjugate comprising:
(i) antibody mAb 3E10 produced by a hybridoma deposited as ATCC accession number PTA-2439, or an antibody having the same binding specificity as an antibody produced by the hybridoma deposited as ATCC accession number PTA-2439, or an antigen binding fragment thereof; and
(ii) human p53 protein comprising SEQ ID NO: 16, or a functional fragment thereof, wherein the cancer cell is a p53-deficient or p53-defective cancer cell, thereby inhibiting or treating metastasis of the cancer cell.

55. The method of claim 54, wherein the antigen binding fragment is selected from the group consisting of Fab, F(ab′)₂, and single chain Fv (scFv) fragments.

56. The method of claim 54, wherein the antigen binding fragment is an scFv fragment of mAb 3E10.

57. The method of claim 54, wherein the administering of the antibody conjugate is by parenteral or intravenous administration.

58. The method of claim 54, wherein the cancer cell is a colorectal cancer.

59. The method of claim 54, wherein the human p53 protein is full-length human p53.

60. The method of claim 54, wherein the p53 protein is a C-terminal fragment.

61. The method of claim 60, wherein the C-terminal fragment comprises amino acids 364-393 of SEQ ID NO:16.

62. A method for restoring p53 function in p53-deficient or p53-defective cancer cells in a subject comprising administering to the subject an antibody conjugate comprising:
(i) antibody mAb 3E10 produced by a hybridoma deposited as ATCC accession number PTA-2439, or an antibody having the same binding specificity as an antibody produced by the hybridoma deposited as ATCC accession number PTA-2439, or an antigen binding fragment thereof; and
(ii) a biologically active molecule capable of restoring p53 function to a p53-deficient or p53 defective cancer cell,
thereby restoring p53 function to the cancer cell.

63. The method of claim 62, wherein the antibody or antigen binding fragment has a light chain having an amino acid sequence at least 95% identical to the amino acid sequence set forth in SEQ ID NO:13.

64. The method of claim 62, wherein the antibody or antigen binding fragment has a heavy chain having an amino acid sequence at least 95% identical to the amino acid sequence set forth in SEQ ID NO:11.

65. The method of claim 62, wherein the restoration of p53 function results in growth arrest, cell cycle arrest, induction of apoptosis, or inhibition or treatment of metastasis.

66. The method of claim 62, wherein the antigen binding fragment is selected from the group consisting of Fab, F(ab′)2, Fv, and single chain Fv (scFv) fragments.

67. The method of claim 66, wherein the scFv fragment comprises the variable region of the heavy chain (VH) and variable region of the kappa light chain (VK) of antibody mAb 3E10.

68. The method of claim 67, wherein the scFv fragment further comprises the signal peptide of the variable region of the kappa light chain (VK) of antibody mAb 3E10.

69. The method of claim 62, wherein the biologically active molecule is a nuclear transcription factor, an enzyme inhibitor, genetic material, an inorganic or organic molecule, a pharmaceutical agent, a drug, or a polypeptide.

70. The method of claim 69, wherein the biologically active molecule is p53 protein or a fragment thereof.

71. The method of claim 62, wherein the antigen binding fragment is an scFv fragment of antibody mAb 3E10 and the biologically active molecule is a p53 protein.

72. The method of claim 71, wherein the p53 protein is human p53.

73. The method of claim 62, wherein the p53 deficiency is selected from the group consisting of an absence of p53, a mutation in p53, and nuclear exclusion of p53.

74. The method of claim 62, wherein the administering of the antibody conjugate is by parenteral or intravenous administration.

75. The method of claim 62, wherein the cancer cell is from a cancer selected from the group consisting of colorectal cancer, esophageal cancer, stomach cancer, leukemia/lymphoma, lung cancer, prostate cancer, uterine cancer, skin cancer, endocrine cancer, urinary cancer, pancreatic cancer, other gastrointestinal cancer, ovarian cancer, cervical cancer, head and neck cancer, bone cancer, kidney cancer, liver cancer, bladder cancer, breast cancer, and adenomas.


SEQUENCE LISTING (18 sequences)

SEQ ID NO: 1 - protein, 5 aa, Artificial sequence
  Synthetic construct linker sequence
GGGGS

SEQ ID NO: 2 - DNA, 24 bp, Artificial sequence
  Primer
ggatccgaggagccgcagtcagat

SEQ ID NO: 3 - DNA, 27 bp, Artificial sequence
  Primer
agatcttcaaatatcgtccggggacag

SEQ ID NO: 4 - DNA, 28 bp, Artificial sequence
  Primer
gaattcgggatggacattgtgctgacac

SEQ ID NO: 5 - DNA, 30 bp, Artificial sequence
  Primer
ccgcggtcaatgatgatgatgatgatggtc

SEQ ID NO: 6 - DNA, 33 bp, Artificial sequence
  Primer
cagtagtcaagtagtaacccctgccttgcacag

SEQ ID NO: 7 - DNA, 34 bp, Artificial sequence
  Primer
catgtattactgtgcaaggcaggggttactactt

SEQ ID NO: 8 - DNA, 43 bp, Artificial sequence
  Primer
gaattcatgcatcatcatcatcatcatgaggagcggcagtcag

SEQ ID NO: 9 - DNA, 24 bp, Artificial sequence
  Primer
ctcgagtcagtctgagtcaggccc

SEQ ID NO: 10 - DNA, 348 bp, Mus musculus
gaggtgcagctggtggagtctgggggaggcttagtgaagcctggagggtcccggaaactctcctgtgcagcctctggattcactttcagtgactatggaatgcactgggtccgtcaggctccagagaaggggctggagtgggttgcatacattagtagtggcagtagtaccatctactatgcagacacagtgaagggccgattcaccatctccagagacaatgccaagaacaccctgttcctgcaaatgaccagtctaaggtctgaggacacagccatgtattactgtgcaaggcgggggttactacttgactactggggccaaggcaccactctcacagtctcctca

SEQ ID NO: 11 - protein, 116 aa, Mus musculus
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 12 - DNA, 333 bp, Mus musculus
gacattgtgctgacacagtctcctgcttccttagctgtatctctggggcagagggccaccatctcctgcagggccagcaaaagtgtcagtacatctagctatagttacatgcactggtaccaacagaaaccaggacagccacccaaactcctcatcaagtatgcatcctacctagaatctggggttcctgccaggttcagtggcagtgggtctgggacagactttcacctcaacatccatcctgtggaggaggaggatgctgcaacatattactgtcagcacagtagggagtttccgtggacgttcggtggaggcaccaagctggagttgaaa

SEQ ID NO: 13 - protein, 111 aa, Mus musculus
DIVLTQSPASLAVSLGQRATISCRASKSVSTSSYSYMHWYQQKPGQPPKLLIKYASYLESGVPARFSGSGSGTDFHLNIHPVEEEDAATYYCQHSREFPWTFGGGTKLELK

SEQ ID NO: 14 - DNA, 321 bp, Mus musculus
agtattgtgatgacccagactcccaaattcctgcctgtatcagcaggagacagggttaccatgacctgcaaggccagtcagagtgtgggtaataatgtagcctggtaccaacagaagccaggacagtctcctaaactgctgatatactatgcatccaatcgctacactggagtccctgatcgcttcactggcagtggatctgggacagatttcactttcaccatcagcagtgtgcaggttgaagacctggcagtttatttctgtcagcagcattatagctctccgtggacgttcggtggaggcaccaagctggaaatcaaa

SEQ ID NO: 15 - protein, 107 aa, Mus musculus
SIVMTQTPKFLPVSAGDRVTMTCKASQSVGNNVAWYQQKPGQSPKLLIYYASNRYTGVPDRFTGSGSGTDFTFTISSVQVEDLAVYFCQQHYSSPWTFGGGTKLEIK

SEQ ID NO: 16 - protein, 393 aa, Homo sapiens [sequence omitted]

SEQ ID NO: 17 - DNA, 1303 bp, Homo sapiens
gtccaggagcaggtagctgctgggctccggggacactttgcgttcgggctgggagcgtgctttccacgacggtgacacgcttccctggattggcagccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcgactcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactga

SEQ ID NO: 18 - protein, 6 aa, Artificial sequence
  Synthetic construct tag sequence
HHHHHH